(19) United States
(12) Patent Application Publication
(10) Pub. No.: US 2003/0133967 A1
(43) Pub. Date: Jul. 17, 2003

(54) Multilayer collagen matrix for tissue reconstruction

(76) Inventors: Zbigniew Ruszczak, Munchen (DE); Robert Mehrl, Langquald (DE); Johann Jeckle, Riedenburg (DE)

Correspondence Address: Synnestvedt & Lechner LLP, 2600 Aramark Tower, 1101 Market Street, Philadelphia, PA, 19107-2950, US

(21) Appl. No.: 10/231,629

(22) Filed: Aug. 30, 2002

Related U.S. Application Data
(63) Continuation-in-part of application No. PCT/EP00/02055, filed on Mar. 9, 2000.

(51) Int. Cl. A61K 9/70; A61F 13/00

(52) U.S. Cl. 424/443

(57) ABSTRACT

A multilayer material with improved mechanical, physical, functional and handling properties for use in human and veterinary medicine for reconstruction of tissue or organs comprises at least two layers of a reconstituted natural polymer matrix, preferentially collagen, and optionally one or more layers comprising a biocompatible synthetic polymer which may be bioinert or biodegradable. The multilayer construct is prepared by simultaneously applying elevated heat and pressure to the combined layers for a short period of time sufficient to bond said layers but insufficient to denature said natural polymer. The materials of the layers may optionally contain biologically active substances or substances which improve mechanical, functional, biological and/or handling properties of the material.

This is a continuation in part application of PCT Application PCT/EP00/02055 designating the US and filed Mar. 9, 2000.

### FIELD OF THE INVENTION

This invention relates to the field of biologically compatible implants, hemostatic agents, wound dressing and sponges, for use in medical applications, including surgical use in human and veterinary medicine for reconstruction of tissue or organs, as well as to a process for the manufacture of such materials. In particular the present invention is concerned with wound healing materials, and in particular with the use of a collagen-containing multilayer membranes for use in surgical applications.

Wound healing dressings and implants should have the ability to adhere and conform to the wound site, and ideally should facilitate regrowth of tissue, and accumulation of fibroblasts, endothelial cells, and wound healing regulatory cells into the wound site to promote connective tissue deposition and angiogenesis and speed healing. The chemical composition and physical characteristics of the implant or dressing are critical to whether these objectives are realized.

Collagen is a major substituent of certain membranes surrounding important organs and separating different tissues and cells, and acts as a superstructure on which cells proliferate, in humans and other animals. Examples of large membranes include the pericardium, peritoneum, intestinal and placental membranes while on the microscopic level, examples include the basal membranes. Consequently, collagen, the major protein of connective tissue, is used in wound dressings and surgical implants

Various different xenogenous, allogenic or autologous collagen-based materials are used in human and veterinary medicine. Purified collagen, even of xenogenous origin, is almost fully biocompatible with human (and also animal of different species) collagenous tissue and may be incorporated into and/or subsequently remodeled to a host connective tissue without foreign body reaction and immunologic rejection. Procedures for rendering xenogeneic collagen substantially non-immunogenic are available. A variety of collagen forms are available including soluble collagen, collagen fibers, collagen processed into sponges, membranes and bone implants. For example, collagen fibers and sponges are used for haemostasis, tissue augmentation and/or as carriers for biologically active substances, collagen membranes are used for wound covering or implantation, as substitutes for missing tissue such as skin, injections of soluble collagen are used in plastic surgery, and multilayer collagen implants based on processed animal large membrane are used for the above applications as well as guided tissue regeneration.

Collagen-based hemostatic agents must have both biological and mechanical features promoting homeostasis such as intact native collagen fibers and optimal porosity. For use as a tissue substitute or equivalent, the collagen-based material must have optimal matrix properties promoting cell growth, formation of granulation tissue, angiogenesis, and vascularization. Collagen-based carriers of biologically active substances must have features allowing an optimal release and pharmacokinetics of the incorporated active substance.

Collagen-based membranes used in surgeries to guide tissue regeneration must have appropriate biological and physical characteristics beyond the few mentioned above. Following surgeries, where wound healing is desirable, undesirable tissue in-growth complicates appropriate tissue regeneration. For example, in dental surgery where a substantial portion of a tooth root is removed, the desired result is the regeneration of healthy bone tissue to replace the bone tissue removed. However, absent appropriate intervention, the cavity left by removal of the bone fills with connective tissue effectively preventing bone regeneration. To prevent this process from delaying healing, a membrane is surgically inserted around the periphery of the wound cavity. This membrane must deter adventitious cell infiltration of the wound cavity and permit the growth of desirable cells.

In all cases, the handling properties of the collagen-based material, including its mechanical strength and stability, its flexibility and, if necessary, its ability to be sutured or sealed are of practical importance.

Reported Developments

Various procedures have been described to improve the mechanical properties of collagen materials. These procedures include additional cross-linking procedures, the most popular of which are chemical cross-linking, for example with aldehydes or temperature initiated cross-linking, and termed “dehydro-thermal treatment”. The aldehyde-based cross-linking is capable of negatively influencing the biocompatibility of collagen and lead to residual aldehyde, or aldehyde derivatives, in the cross-linked product.

U.S. Pat. No. 3,157,524, discloses a sponge comprised of acid treated swollen collagen. Oluwasanmi et al. (J. Trauma 16:348-353 (1976)) discloses a 1.7-millimeter thick collagen sponge that is cross-linked by glutaraldehyde. Collins et al. (Surg. Forum 27:551-553 (1976)) discloses an acid-swollen collagen sponge that is cross-linked by glutaraldehyde. U.S. Pat. No. 4,320,201 discloses a swollen sponge of high collagen purity produced by enzymatically degrading animal hides, digesting the mass in alkali or acid, mechanically comminuting the mass to produce specified lengths of collagen fibers, and cross linking the fibers. U.S. Pat. No. 4,837,285 discloses porous beads that have a collagen skeleton of 1 to 30 percent of the bead volume. These beads are useful as substrates for cell growth. In addition, collagen has been used as a component in salves (PCT Patent Application WO 86/03122). U.S. Pat. No.4,937,323 discloses the use of collagen for wound healing in conjunction with electrical currents. Abbenhaus et al., Surg. Forum 16:477-478 (1965) discloses collagen films of two to three millimeter thickness that were produced by heating and dehydrating collagen extracted from cow hides. U.S. Pat. No. 4,412,947 discloses an essentially pure collagen sheet made by freeze drying a suspension of collagen in an organic acid. British Patent 1,347,582 discloses a collagen wound dressing consisting of a freeze dried polydisperse collagen mixture. U.S. Pat. No. 4,950,699 discloses a wound dressing consisting of less than 10% collagen mixed with an acrylic adhesive.

European Patent Application 187014, U.S. Pat. No. 4,600,533,; U.S. Pat. No. 4,655,980; U.S. Pat. No. 4,689,399; and PCT Patent Application WO 90/00060 disclose non-chemically cross linked collagen implants produced by compression, which are useful for sustained drug delivery. U.S. Pat. No. 4,453,939 discloses a wound-healing composition containing collagen coated with fibrinogen, factor XIII fibrinogen, and/or thrombin. U.S. Pat. No. 4,808,402 discloses a composition for treating wounds comprising collagen, bioerodible polymer, and tumor necrosis factor. U.S. Pat. No. 4,703,108 discloses that fibronectin, laminin, type IV collagen and complexes of hyaluronate and proteoglycans may be included in a collagen-based matrix, having a swelling ratio of between 2.5 to 5 for collagen-based matrices that comes into contact with open wounds, or a swelling ratio of between 2.5 to 10 for collagen-based matrices for subcutaneous implantation. The thickness of the collagen-based matrix is varied from 1 to several hundred mm, and preferably between 2 to 3 mm for full thickness wound dressings.

Commercially collagen-based materials are available in the form of sponges, transparent membranes, multilayer animal membrane based products, and injectable solutions of varying viscosities. Collagen-based sponges and membranes are used for tissue substitution, haemostasis, skin substitution and as a carrier for biologically active substances. The Collatamp®-G product, manufactured by SYNTACOLL AG, Herisau, Switzerland, is sold and distributed worldwide by Schering-Plough (USA) and its Essex Chemie subsidiary is the only commercially available collagen-based drug delivery system for antibiotics.

All currently available collagen-based materials are, however, not stable enough to be sutured, rolled, or stitched, especially in areas of mechanical tension or in difficult anatomical sites. Moreover, collagen sponges or membranes are—in many cases—not strong enough to sufficiently cover defects of such tissue as, i.e., dura mater, superficial and deep skin wounds, bones, nerves, etc.

The use of defined mechanical pressure for industrial manufacture of collagen membrane-like products based on freeze-dried collagen sponges containing active substances, i.e. antibiotics like gentamycin, is known per se (see EP 0 069 260, issued Sep. 25, 1985, owned by Syntacoll AG, Herisau, Switzerland).

U.S. Pat. No. 4,522,753 describes a method for preserving porosity and improving stability of collagen sponges by both aldehyde and dehydro-thermal treatment. The negative pressure (vacuum) used in this process may vary from about 1 mtorr up to a slight vacuum below atmospheric pressure.

U.S. Pat. No.4,578,067 describes a hemostatic-adhesive collagen dressing in the form of a dry-laid, non-woven, self-supporting web of collagen fiber. The manufacturing of such material is based on a Rando-feeder and Rando-webber techniques. The collagen fibers from the Rando-feeder are introduced into the air stream of the Rando-webber and form a fiber mass of uniform density. Such mass is then processed by pressing or embossing or by calendaring at a temperature ranging from room temperature to 95° C. The inherent limitation of such techniques is that the pressures to which the fiber mass is subjected are limited to preparing relatively thick layers of material of relatively low density.

The U.S. Pat. No. 5,206,028 describes a collagen membrane having improved physical and biological properties. Such membrane does not swell appreciably upon being wetted and maintains its density. The translucent, collagen Type-1 based material is prepared by the compression of collagen sponges, at atmospheric pressure and ambient temperature, followed by chemical cross-linking to set the swellability of the sponge to a fraction of the compressed volume. For additional mechanical stabilization, the cross-linked membrane may be re-wetted, re-lyophilized, and pressed again under standard condition. The '028 patent specifically discloses the use of a roller press with a calibrate aperture followed by aldehyde cross-linking. The compressed sponge is disclosed to desirably have a bulk density in the range of from about 0.5 to about 1.5 g/cc, and preferably in the range of from about 0.8 to about 1.2 g/cc, and that from an initial sponge height of from about 7 mm to about 13 mm, compressed sponges are reduced to a height of from about 0.15 mm to about 0.25 mm, preferably to a height of from about 0.17 mm to about 0.23 mm.

U.S. Pat. No. 4,948,540 describes a mechanically stable, collagen wound dressing sheet material fabricated by lyophilizing a collagen composition and compressing the porous pad at a pressure between about 15,000 and 30,000 p.s.i to a thickness of between 0.1 to 0.5 centimeters at a pressure to yield a collagen dressing sheet material having an absorbability of 15-20 times its weight. The '540 patent also discloses that the material may be cross-linked by dehydro-thermal treatment to improve mechanical stability.

U.S. Pat. No. 4,655,980 discloses the manufacturing of collagen membrane articles based on a soluble collagen gel suspension. The membrane may be obtained by applying pressure to the gel, or by disrupting the gel and separating the resulting precipitate for casting. Depending on the dimension and shape of the casting mold, either a membrane or solid can be obtained. The manufacturing of such membrane is based on a commercially available soluble, injectable, atelocollagen product of Collagen Aesthetics, Palo Alto, Calif., USA.

Artificial collagen-containing multilayer membranes have been described in the prior cut and proposed for the dressing or coverage of wounds. Yannas and Burke (J. Biomed. Mat. Res. 14:68-81 (1980) have reviewed the design of artificial skin, some examples of which contain collagen. European Patent Application 167828 and U.S. Pat. No. 4,642,118 disclose an artificial skin composed of two layers: collagen and a poly-alpha-amino acid. U.S. Pat. No. 4,841,962 discloses a wound dressing composed of three layers: an adhesive, a cross-linked collagen matrix, and a multilayer polymer film. U.S. Pat. No. 5,512,301 discloses collagen-containing sponges comprising an absorbable gelatin sponge, collagen, and an active ingredient to deliver pharmaceutically active substances over extended periods of time. WO-A-88/08305 (The Regents of The University of California) discloses a composite skin replacement, which consists of a layer of human epidermal cells together with a layer of a biosynthetic membrane, which may be formed of collagen and mucopolysaccharides. However, the collagen/-mucopolysaccharide portion is of uniform texture throughout, is strongly immuno-reactive, and can only be used on the donor of the cells. Another artificial collagen-containing membrane is described in DE-A-2631909 (Massachusetts Institute of Technology). This membrane consists of a minimum of two layers, the first layer being a combination of collagen and mucopolysaccharides and the second layer being a synthetic polymer such as a polyacrylate. However, this membrane is totally non-resorbable, the collagenous layer being so tightly cross-linked internally that resorption cannot occur.

U.S. Pat. No. 5,219,576 and WO99/19005 describe a collagen implant material useful as a wound healing matrix and delivery system for bioactive agents. The '576 patent discloses the manufacturing of multilayer collagen materials by serially casting and freezing the individual layers and then lyophilizing the entire composite at once. Additional cross-linking by both aldehyde and dehydro-thermal processing of the final product is also disclosed. The '576 patent discloses compressing the single layer implants from a thickness of 5 mm to 1 mm to increase its bulk density. The '576 patent discloses that compressed implants typically have bulk densities in the range of 0.05 to 0.3 g/cc, whereas non-compressed implants normally have bulk densities of 0.01 to 0.05 g/cc. The '576 patent does not suggest simultaneous heat curing and compression.

U.S. Pat. No. 5,733,337 discloses a multilayer tissue repair prosthesis comprising two or more superimposed, bonded layers of collagenous tissue material sourced from the tunica submucosa of the small intestine, fascia lata, dura mater, or pericardium, wherein the layers are bonded together by heat welding from about 50° C. to about 75° C. from about 7 minutes to about 24 hours, typically about one hour, and wherein said prosthesis is cross linked with a cross linking agent that permits bioremodeling. The bonding of the collagen layers may be accomplished in a number of different ways: by heat welding, adhesives, chemical linking, or sutures.

U.S. Pat. No. 6,206,931 and WO98/22158 disclose graft prostheses including a purified, collagen-based matrix structure removed from a submucosa tissue source. Multiple layer structures of the treated submucosa are disclosed but no method of lamination other than folding or stapling suggested.

The types of membrane used to guide tissue regeneration include synthetic, non-resorbable membranes, such as Gore-Tex (trade mark); synthetic resorbable membranes formed from glycolide and lactide copolymers, and resorbable collagen-containing multilayer membranes based on intact processed animal large membrane, such as peritoneum. The non-collagen-containing membranes are disadvantageous due to the need to surgically remove the membrane, the generation of irritant breakdown products, and/or the lack of hemostatic and cell in-growth properties to promote wound healing.

The membranes disclosed in U.S. Pat. No. 5,837,278 is derived directly from naturally occurring membranes, which, as far as possible, retain their natural collagen structure. The disclosed preferred source of membrane is the naturally occurring peritoneum membrane, especially taken from calves or piglets. The collagen-containing large membrane products rely on the processing and recovery of a large number of intact animal membranes, the properties of which may differ from animal to animal.

There is a need, in both human and veterinary medicine, to create biopolymer-based and particularly collagen-based multilayer materials with enhanced mechanical and physical properties that can be prepared from reconstituted biopolymer, to reduce the cost of, and improve, large scale commercial production of such materials.

Moreover, there is a need to create collagen-based multilayer constructs in which collagen components can be joined on physical and/or mechanical basis without additional cross-linking substances of potential negative value for living cells or tissue, by increasing immunologic sensitivity resulting in foreign body reaction or granuloma formation.

There is also a need to create collagen-based multilayer materials, in which each layer exhibits a pre-determined mechanical, physical and/or physiologic properties, such as wetting, fluid absorption and/or remodeling/degradation times, wet tensile and suture strengths, which are reproducible on a large scale and which can function as long term tissue implants and substitutes, having slow bio-degradation and bio-incorporation rates.

The present invention responds to these needs and provides a multilayer comprising a reconstituted biopolymer material with the aforesaid properties and advantages.

As will be described in detail below, the present invention combines heat and positive mechanical pressure, both known individually for use by the skilled worker, for the treatment of the reconstituted materials according to the present invention. The influence of a moderate heat, especially if used together with a negative pressure (vacuum), for induction of additional cross-linking sites in collagen sponges has been described previously as dehydro-thermal treatment (see above). However, the prior art neither discloses nor suggests such a technical combination, which technique enables the manufacture of a diverse and flexible range of multilayer biocompatible products with highly unexpected, superior properties, as will be described in more detail below.

### SUMMARY OF THE INVENTION

The present invention provides a multilayer biocompatible sheet material comprising a first and second layer comprising reconstituted matrices of biocompatible collagen, which layers are physically adhered along at least a portion of a surface of each of said layers, wherein said material has sufficient flexibility to form tubes useful for tissue and organ reconstruction, and wherein at least one said layer is capable of absorbing sufficient fluids to form an expanded matrix capable of promoting cell growth. A particularly preferred embodiment of the present sheet invention is wherein said matrix is porous and capable of promoting formation of granulation tissue, angiogenesis, and vascularization.

The reconstituted collagen used in the present invention exhibits hemostatic adhesive properties of native collagen, is non-antigenic, and is used in the form of spongy layers containing pores of biologically functional size, and compressed spongy layers having expansion capacities on contact with aqueous fluids of from less than a fraction of a volume to the full volume of an uncompressed sponge. The individual layers of the multilayer sheet are selected from reconstituted biopolymer sheets having specific densities and result in strongly adherent multilayer sheets that have properties controlled by the parameters of the present lamination process.

The present invention further provides a process for the preparation of a multilayer biocompatible sheet material having sufficient flexibility to form tubes useful for tissue and organ reconstruction, comprising

aligning the surface of a first sheet of reconstituted matrix of biocompatible collagen with the surface of a second sheet of reconstituted matrix of biocompatible collagen to form a non-adherent bilayer construct, and

applying mechanical pressure and elevated temperature simultaneously and uniformly along at least a portion of the surface of said construct for a time sufficient to adhere said layers but insufficient to denature said collagen.

The present invention also provides a multilayer biocompatible sheet material having sufficient flexibility to form tubes useful for tissue and organ reconstruction prepared according to the aforesaid process. The present invention may be used in human and veterinary medicine, for in vivo applications such as wound healing dressings, implants, directed tissue regeneration, the reconstruction of tissues and organs, and ex vivo cell growth.

Additional and preferred aspects and embodiments of the present invention are described in more detail below.

### DETAILED DESCRIPTION

The terms defined in this section are used throughout this specification.

The term “antibiotic” as used herein means a substance produced synthetically or isolated from natural sources that selectively inhibits the growth of a microorganism.

The term “biocompatible” as used herein means the ability of a material to pass the biocompatibility tests set forth in International Standards Organization (ISO) Standard No. 10993 and/or the U.S. Pharmacopoeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” These tests assay for a material's toxicity, infectivity, pyrogenicity, irritation potential, reactivity, hemolytic activity, carcinogenicity, and/or immunogenicity. A biocompatible composite, or polymer comprising a layer thereof, when introduced into a majority of patients will not cause an adverse reaction or response. In addition, it is contemplated that biocompatibility can be effected by other contaminants such as prions, surfactants, oligonucleotides, and other biocompatibility effecting agents or contaminants.

The term “bioinert” as used in relation to a material means a material that does not interact with biological systems. A “bio-inert” material is non-reactive with the components of blood, and tissues, including the immunological and coagulation systems. Bioinert substances neither initiate coagulation nor raise an immunological response in host tissue, and the chemical make-up of such substances is not altered while in contact with such biological systems.

The term “contaminant” as used herein means an unwanted substance on, attached to, or within a material, such a layer of the present composite. This includes, but is not limited to bioburden, endotoxins, processing agents such as antimicrobial agents, blood, blood components, viruses, DNA, RNA, spores, fragments of unwanted tissue layers, cellular debris, and mucosa.

The term “cells” as used herein means a single unit biological organism that may be eukaryotic or prokaryotic. The eukaryotic cell family includes yeasts and animal cells, including mammalian and human cells. Cells that may be useful in conjunction with the present invention include cells that may be obtained from a patient, or a matched donor, and used to seed a wound site. Such seeding would be used in an effort to repopulate the wound area with specialized cells, such as dermal, epidermal, epithelial, muscle or other cells, or alternatively to provide cells those stimulates or are involved in providing immunological protection to fight off infectious organisms. Such cells may be isolated and extracted from the patient, and/or genetically reengineered to produce a host of cytokines, antibodies, or other growth factors to aid in the wound healing process.

The term “composite” as used herein means a solid material which is composed of two or more substances having different physical characteristics and in which each substance retains its identity while contributing desirable properties to the whole.

The term “cytokine” as used herein means a small protein released by cells that has a specific effect on the interactions between cells, on communications between cells or on the behavior of cells. The cytokines includes the interleukins, lymphokines, and cell signal molecules, such as tumor necrosis factor and the interferons, which trigger inflammation and respond to infections. Many cytokines are produced by recombinant technology and are presently available for use in research as well as by prescription in human and animal subjects.

The term “growth factor” as used herein means a substance (as a vitamin B₁₂ or an interleukin) that promotes growth and especially cellular growth. Examples of growth factors include, but are not limited to, epidermal growth factor, which is a polypeptide hormone that stimulates cell proliferation, nerve growth factor, which is a protein that promotes development of the sensory and sympathetic nervous systems and is required for maintenance of sympathetic neurons, vascular endothelial growth factors, which are a family of proteins that stimulate angiogenesis by promoting the growth of vascular endothelial cells, and the like. The term “oncostatically effective amount” is that amount of growth factor that is capable of inhibiting tumor cell growth in a subject having tumor cells sensitive to the selected agent. For example, many non-myeloid carcinomas are sensitive to treatment with TGF-beta, particularly TGF-beta2. The term “hematopoietically modulatory amount” is that amount of growth factor that enhances or inhibits the production and/or maturation of blood cells. For example, erythropoietin is known to exhibit an enhancing activity at known dosages, while TGF-beta exhibits an inhibitory effect. The term “osteoinductive amount” of a biological growth factor is that amount which causes or contributes to a measurable increase in bone growth, or rate of bone growth.

The term “medicament” as used herein means a substance used in medical therapy, such as the therapeutically effective active ingredient in a pharmaceutical. The term “immunomodulatory amount” of a medicament or agent is an amount of a particular agent sufficient to show a demonstrable effect on the subject's immune system. Typically, immunomodulation is employed to suppress the immune system, e.g., following an organ transplant, or for treatment of autoimmune disease (e.g., lupus, autoimmune arthritis, autoimmune diabetes, etc.). For example, when transplanting an organ one could line the site with the matrix of the invention impregnated with an immunomodulatory amount of an immunosuppressive biological growth factor to help suppress rejection of the transplanted organ by the immune system. Alternatively, immunomodulation may enhance the immune system, for example, in the treatment of cancer or serious infection (e.g., by administration of TNF, IFNs, etc.).

The term “membrane” as used herein means a thin soft pliable sheet or layer. The term “natural polymer” as used herein means a polymer that is found in nature and that may be derived from natural sources or produced synthetically. More particularly, the natural polymer means a polymer comprising repeating subunits of small organic molecules found in biological systems including microorganisms, plants, and animals. Exemplary subunit molecules include the groups of molecules known as the nucleotides, amino acids, and saccharide molecules. Polymers containing these small molecules comprise the polynucleic acids, such as the polyribonucleic acids and the polydeoxyribonucleic acids, the polypeptides, such as the proteins such as the structural proteins collagen and keratin, and small polypeptides comprising certain hormones and other signaling molecules, and polysaccharides, such as the cellulose and alginic acid family of molecules, respectively.

Preferred natural polymers exhibit properties similar to collagen and are useful for the same applications. Examples of such substances are collagen, gelatin, and hyaluronic acid. Collagen is the more preferred natural polymer.

The collagen used for manufacture of the collagen-based multilayer materials of the present invention may be either of animal origin (xenogenous to humans) or human origin (autologous or allogenic) or may be obtained from genetically manipulated organisms (recombinant techniques and/or transgenic organisms), or by any other similar or/and equivalent method. The collagen used for manufacturing of the improved collagen-based material may be of Type-l, Type-ll, Type-Ill, Type-IV, Type-VII, or Type-IX alone or may be a mixture of two or more of such collagens. The more preferred collagen used for manufacture of the present collagen-based multilayer product is Type-1 collagen. This material can be easily obtained from animal tissue, such as skin, tendons, and membranes, by industrial methods know to the person of skill in the art, in accordance with GMP standards of manufacturing.

The present invention may use enzymatically treated collagen or collagen that has not been enzymatically treated. Preferred collagen is enzymatically treated with proteolytic enzymes, to separate the non-helical parts of the collagen molecule (telopeptides) from the triple-helical collagen chain (atelocollagen).

The term ‘reconstituted” as used herein describes a material that has its origin in a solid source or form such as a solid matrix, that has been disrupted by chemical, physical or biological processes, that may have been dispersed or dissolved in a liquid medium, and that has been reformed, or restructured, into a further solid form having a structure that is modified physically and/or chemically relative to the original solid form of the material.

The reconstituted natural polymer matrices and/or the synthetic polymer material layer may optionally contain biologically active substances such as hemostatic agents, such as polysaccharides and glycosaminogtycans, proteins, such as cytokines and growth factors and hormones, cells or cell extracts medicaments, such as antibiotics, anti-inflammatory agents, or biologically important and tissue-compatible inorganic or/and organic substances or/and their derivatives which can improve the mechanical, functional, biological and handling properties of the material. Exemplary antibiotics include but are not limited to gentamycin, tetracycline, doxycycline, teicoplanin, quinoline antibiotics including the fluroquinolones, vancomycin, synercid®, penicillin derivatives and the cephlosporins. One or more protein agents may be incorporated to promote granulation tissue deposition, angiogenesis, re-epithelialization, and fibroplasia. Additionally, these and other factors are known to be effective immunomodulators (either locally or systemically), hematopoietic modulators, osteoinductive agents, and oncostatic agents (e.g., TGF-beta has been shown to exhibit all of these activities). The bioactive additives or protein factors used herein may be native or synthetic (recombinant), and may be of human or other mammalian type. Human FGF (including both acidic or basic forms), PDGF, and TGF-beta are preferred. Methods for isolating FGF from native sources (e.g., pituitary, brain tissue) are described in Bohlen et al, Proc Nat Acad Sci USA, (1984) 81:5364, and methods for isolating PDGF from platelets are described by Rainer et al, J Biol Chem (1982) 257:5154. Kelly et al, EMBO J (1985) 4:3399 discloses procedures for making recombinant forms of PDGF. Methods for isolating TGF-beta from human sources (platelets and placenta) are described by Frolik et al in EPO 128,849 (Dec. 19, 1984). Methods for isolating TGF-beta and TGF-beta2 from bovine sources are described by Seyedin et al, EPO 169,016 (Jan. 22, 1986), and U.S. Ser. No. 129,864, incorporated herein by reference. Other exemplary agents include, without limitation, transforming growth factor-alpha, beta-thromboglobulin, insulin-like growth factors (IGFs), tumor necrosis factors (TNFs), interleukins (e.g., IL-1, IL-2, etc.), colony stimulating factors (e.g., G-CSF, GM-CSF, erythropoietin, etc.), nerve growth factor (NGF), and interferons (e.g., IFN-alpha, IFN-beta, IFN-gamma, etc.). Synthetic analogs of the factors, including small molecular weight domains, may be used provided they exhibit substantially the same type of activity as the native molecule. Such analogs are intended to be within the scope of the term “wound healing agent,” as well as within the specific terms used to denote particular factors, e.g., “FGF,” “PDGF,” and “TGF-beta.” Such analogs may be made by conventional genetic engineering techniques, such as via expression of synthetic genes or by expression of genes altered by site-specific mutagenesis. Factors, such as with PDGF, may be incorporated into the native polymer layer in its native form (i.e., in platelets), or as crude or partially purified releasates or extracts. Alternatively, the factors may be incorporated in a substantially pure form free of significant amounts of other contaminating materials.

Such additional agents are included in the reconstituted natural polymer layer in therapeutically effective local concentration amounts. The amount of the agent included in the composite of the present invention will depend upon the particular agent involved, its specific activity, the type of condition to be treated, the age and condition of the subject, the severity of the condition and intended therapeutic effect. For example, it may be necessary to administer a higher dosage of a factor when using the composite to treat a wound resulting from surgical excision of a tumor, than when simply promoting the healing of a wound (e.g., due to trauma or surgical procedure). In most instances, the protein factor(s) will be present in amounts in the range of about 3 ng/mg to 30 ug/mg based on weight of collagen. Antibiotic agents, such as gentamycin, are present in amounts that range from about 100 microgram/cm3 to about 1×10 microgram/cm3.

The agents may be added to the multilayer materials of the present invention after the adhesion thereof, or preferably are added during the manufacture of the reconstituted matrices prior to the removal of the medium by evaporation or flash freezing and lypholisation. Alternatively, such materials, with or without additional carrier materials such as collagen fibers, may be included during the adhering process of sandwiches or tortellini constructs as described in more detail below.

The present invention provides multilayer composite materials including reconstituted natural polymer matrices, and preferably collagen-based matrices, that are biocompatible, resorbable, and that exhibit improved and variable, but defined, mechanical stability, dry and wet tension, fluid absorption, and flexibility.

The preferred sheet material layers according to the present invention comprise collagen that exhibits the hemostatic and non-antigenic properties of native collagen. Such collagen is biocompatible, biodegradable, and resorbable.

The multilayer construct of the present invention may combine different forms of reconstituted collagen matrix, including freeze-dried sponges; air-dried membranes, freeze-dried pre-pressed sponges, and chemically modified collagen matrices, such as cross-linked matrices, and antigenically modified collagen matrices. The term “sponge” as used herein means an elastic porous mass of interlacing fibers that is able when wetted to absorb water. The term “pore” as used herein means a small interstice admitting the absorption or passage of liquid or a cell. “Porous” means a material containing pores.

A preferred aspect of the sheet material according to the present invention is where each of at least two layers comprises a compressed sponge or transparent membrane of reconstituted collagen.

A further special embodiment of the present multilayer sheet invention further comprises a third layer comprising a biocompatible synthetic polymer. The term “synthetic polymer” as used herein means a polymer comprising repeating subunits of small organic molecules that are not found in natural biological systems. Exemplary subunit molecules include the urethanes, esters, ethers, silicones, vinyl alcohols, halovinyl alcohols, amides, fluorinated alkanes, styrene, and halogenated arylenes. Foams of synthetic polymers are preferred materials for use in such layers. The term “foam” as used herein means a solid material, throughout which are distributed voids, pores or cells, which are at least partially open and function to interconnect the voids throughout the material. Foam materials may be produced from a polymerization mixture containing gas-generating agents of through which gas is pumped during the polymer solidification process.

A further preferred aspect of the present sheet material invention is that its constituent layers remain adhered to each other when placed into contact with blood and/or tissue fluids under physiological conditions, for at least as long required for cells to infiltrate and populate the interlayer boundary. This can take anywhere from about a day to a week for the cell migration and in-growth process to influence and diminish interlayer boundary distinctions. Of course, the specific characteristics of the layers comprising the multilayer composite will greatly influence the amount, direction, speed, and degree of cell ingrowth, and resulting biodegradation and tissue incorporation. Prior to use, the adhesion of the layers is provided by the inherent adhesive nature of the collagen comprising the reconstituted matrix of the layer, and is free of any additional adhesive material or agent. A most preferred embodiment of the present sheet material exhibits an adhesive strength between said layers that is about equal to or greater than the wet tensile strength of each of said layers.

Another aspect of the present invention is a multilayer sheet material having a wet tensile strength such that the material may be handled wet during surgical procedures and sutured without tearing under normal conditions of use. A more preferred multilayer material exhibits a wet tensile strength of about 5 N to about 25 N.

A particularly preferred multilayer sheet material according to the present invention includes a first layer that is capable of expanding about 3 to about 10 times in volume on contact with fluids. Such expanded reconstituted layers provide for pore sizes that permit the passage of cells that contribute to tissue regeneration and wound healing. A specific embodiment of the present invention includes a second layer that is capable of expanding less than about 3 times in volume on contact with fluids. Such expanded reconstituted layers provide for pore sizes that permit the passage of fluid but not large numbers of cells. The smaller the fluid expansion, the smaller the pore size, and the more impenetrable the particular layer will be to the passage of cells and biological fluids.

Preferred reconstituted collagen layers of the present sheet material include a first and second layers that have a thickness of less than about 1 mm, and a density of about 250 mg/cc to about 500 mg/cc, and preferably about 260 mg/cc to about 300 mg/cc, and most preferably about 270 mg/cc to about 290 mg/cc. Another special embodiment of the present multilayer invention is where said first and second layers have substantially the same dry thickness. A further embodiment of the present sheet material according to the present invention is where said first and second layers have substantially different dry thicknesses and densities. More preferred aspects include first layers having thicknesses of from about 0.1 to about 0.6 mm. Preferred second and reconstituted layers preferably have a thickness less than about 1 mm, preferably from about 0.05 mm to about 0.1 mm.

The present invention includes collagen-based multilayer constructs of leather-like collagen sheets of different strength, collagen “pockets” or “tortellini-Like” constructs, collagen “sandwich”-like structures of different permeability and porosity as well as collagen tubes and channels with or without a lumen. In the latter case, the “lumen” of the tube or channel may be filled with a core of collagen-based material of various densities and/or porosities.

A particularly preferred embodiment of the present invention is a tubular article having an inner and outer surface, comprising a sheet material according to the present invention wherein the aforesaid first layer that is fluid expandable from about 3 to about 10 times comprises the inner surface of the tube. A more preferred tubular article according to the present invention has an outer surface that comprises a said second layer that expands less than about 2 times in volume on contact with fluids. A most preferred tubular article according to the present invention is wherein said outer surface is water permeable and substantially non-porous.

The tortellini and sandwich constructions referred to above include, in the interiors of the multilayer constructs, materials such as biologically active substances, as described in more detail above.

The present invention provides multilayers of at least two layers of the same or different materials, that are preferably all reconstituted natural polymer, more preferably, collagen based matrices, or a combination of at least two reconstituted natural polymer layers, with one or more additional layers, that may be reconstituted natural polymer, preferably collagen, or one or more synthetic polymer layer that may be bioinert or biodegradable, preferably comprising a silicone polymer.

A special embodiment of the present multilayer sheet invention includes, in addition to the first and second layers of reconstituted collagen, from one to four additional layers each comprising a reconstituted matrix of biocompatible collagen.

The present multilayer invention exhibits excellent mechanical properties, especially dry and wet tension, natural hemostatic properties, improved wetting abilities, and can be rolled or screwed in dry or wet condition without loosing shape or disintegrating. Moreover, through application of the present processing invention, the multilayer's permeability for air (or other gases) and water (or other fluids, including blood, or tissue fluids) as well as mechanical strength can easily be controlled due to variations in the manufacturing process. The variations of the processing parameters to achieve such varying properties are known to the skilled artisan.

The present invention enables the permanent or temporary lamination of different reconstituted natural polymer matrices, preferably collagen-containing matrix products or layers, into a multilayer product by simultaneously applying defined mechanical pressure and defined heat to at least two product layers under conditions that protect the fibrilar native (and/or re-natured) structure of the reconstituted collagen from degradation or/and denaturation or/and melting and which preserves the natural biologic properties of collagen, including the hemostatic properties and cell growth-promoting matrix properties.

The present invention therefore relates to a process for the preparation of a multilayer biocompatible sheet material having sufficient flexibility to form tubes useful for tissue and organ reconstruction, comprising aligning the surface of at least a portion of a first sheet of reconstituted matrix of biocompatible collagen with the surface of a second sheet of reconstituted matrix of biocompatible collagen to form a non-adherent bilayer construct, and applying mechanical pressure and elevated temperature simultaneously and uniformly along the entirety of said contacting surfaces of said construct for a time sufficient to adhere said layers but insufficient to denature said collagen. A preferred process contacts two or more layers along the entire length and width of the adjacent surfaces of each of said layers.

The temperature used in the present process is in a range of from about 50° C. to about 200° C., while the pressure used is in a range of from about 0.1 to about 1000 kg/cm². The time during which the thermal compression is administered to the multilayer construct is between 0.1 second to about one hour. A preferred process according to the present invention employs a pressure of about 5 to about 10 kg/cm2, an elevated temperature from about 50 to about 120° C., and a thermal compression time of about one to about 60 seconds. A more preferred thermal compression time is from about ten to about 30 seconds, and a most preferred time is about 10 to about 20 seconds.

The treatment can be conducted in a conventional thermal pressing machine in which the parts exerting the pressure can be adjusted to a predefined and constant temperature. The manufacturing steps used for the preparation of the novel multilayer material can be easily incorporated into routine manufacturing processes and allows the savings of time and costs compared to other currently used methods used for the production of collagen products.

As a result of such a heat and pressure treatment, a collagen-containing membrane-like structure of desired thickness, mechanical strength, permeability, degradation and resorption time, can be manufactured. Moreover, the manufactured product exhibits much better handling properties than other known collagen-based products such as freeze-dried sponges or air-dried membranes.

To obtain the novel multilayer membrane-like material of appropriate mechanical and physiological properties, different layers of reconstituted natural polymer, preferentially pre-pressed collagen membranes, non-pre-pressed collagen sponges or air-dried collagen membranes alone or in different combinations may be used. The basic material for manufacturing the novel multilayer material is preferably a pre-pressed, non-transparent collagen sponge, or an uncompressed collagen sponge, a transparent collagen membrane or a combination of these products. The present process according to the invention uses a first sheet that comprises a biocompatible collagen sponge having a thickness of about 1 to about 10 mm, and a density of about 2 to about 60 mg/cc. One aspect of the present process uses a second layer according to the present invention comprises a biocompatible collagen sponge having a thickness of about 1 to about 10 mm, and a density of about 2 to about 60 mg/cc. Another aspect of the process according to the present invention uses a second layer that comprises a biocompatible transparent collagen membrane having a thickness of about 0.01 to about 0.1 mm.

A further aspect of the process according to the present invention provides for one or more additional layers of biocompatible reconstituted collagen matrix that are aligned with said first and second layers to form a non-adherent construct. Such further layers may are aligned subsequent to the application of heat and pressure to said first and second layers.

The process is conducted under temperature and pressure parameters such that result in the production of a multilayer biocompatible sheet material having sufficient flexibility to form tubes useful for tissue and organ reconstruction.

A further aspect of the present invention is a multilayer sheet material prepared by the present process invention wherein the thickness of said first layer is reduced to about 1 to about 30 percent of its original thickness. A preferred embodiment of the process is the preparation of a multilayer sheet material where the thickness of said first layer is reduced to about 1 to about 3 percent of its original thickness, and is capable of absorbing about 3 to about 7 times its weight in fluids in about sixty seconds.

A more preferred embodiment is where the thickness of said first layer is reduced to about 5 to about 30 percent of its original thickness, and is capable of absorbing about 4 to about 30 times its weight in fluids in about sixty seconds. A most preferred embodiment according to the present invention is wherein the thickness of said first layer is reduced to about 6 to about 25 percent of its original thickness, and is capable of absorbing about 25 to about 30 times its weight in fluids in about sixty seconds.

The collagen sponge may be manufactured using various state-of-the-art techniques. The basis for such a material may be collagen dispersion/suspension (i.e. in water or other non-organic solvent) of 0.5 to 5.0 weight % of dry collagen. The sponge is prepared preferably by freeze-drying.

A special embodiment of the multilayer material according to the present invention may be prepared from reconstituted collagen layers that have been previously simultaneously treated with defined heat and pressure. Such layer may be described as a non-transparent, membrane-like structure.

A transparent collagen membrane may be manufactured using different state-of-the-art techniques. The basis for such a material may also be collagen dispersion/suspension (i.e. in water or other non-organic solvent) of 0.5 to 5.0 weight % of dry collagen. The membrane will preferably be obtained by controlled air-drying. Such preformed membranes are preferably used in special embodiments of the present invention.

Still another subject of the present invention is the use of the novel multilayer material of the invention for the medical indications and applications mentioned above.

The use of different densities, and forms, of collagen sponges or collagen membranes will influence the mechanical properties and biological function, in particular, the remodeling/degradation ratio, of the multilayer material of the present invention. Moreover, the bio-degradation and active agent release ratios are likely to be influenced and controlled by the relative placement and characteristics of the individual collagen matrix layers (pre-compressed or not pre-compressed) as well as various additional ingredients (i.e. biologically active substances) incorporated into the collagen matrix.

The invention will be further described and illustrated by the following examples.

### EXAMPLES

In these following examples, the manufacture of the simplest multilayer sheet materials comprising two or three similar or different basic reconstituted matrix layers (sponges or/and membranes) is described.

### Example 1

### Manufacture of a Collagen-based Membrane-like Bilayer Material from Two Uncompressed Sponges.

Two freeze-dried collagen sponges (Collatamp®, manufacturer: SYNTACOLL AG, Herisau, Switzerland) are conditioned at 21° C. in a moisture chamber to water content of 14%. After conditioning, the sponges are placed one above the other to form a double-layer and prepared for thermo-mechanical pressing. Continuous heat of 100° C. and pressure of 25 kg/cm² is applied to the sponges for 10 seconds to form a double-layer construct. After pressing, the surfaces of the press are opened without pre-cooling. The collagen-based double-layer membrane obtained is not transparent. It has an excellent mechanical stability, flexibility, good fluid absorption, and good hemostatic properties. The collagen layers are joined physically, the mechanical stability of the junction is very high. The material obtained can be used in both ex vivo and in vivo medical applications.

### Example 2

### Manufacture of a Collagen-based Membrane-Like Bilayer Material (Pre-Pressed Sponges).

Two freeze-dried collagen sponges (Collatamp®, manufacturer: SYNTACOLL AG, Herisau, Switzerland) are pressed separately as in Example 1. After pressing, the membranes are conditioned to water content of 10-15% and placed one above the other to form a double-layer and prepared for thermo-mechanical pressing. Continuous heat of 100° C. and pressure of 25 kg/cm² is applied to the sponges for 10 seconds to form a new double-layer construct. Each layer of this material consists of two previously pressed sponges of defined porosity.

After finishing pressing, the surfaces of the press are opened without pre-cooling. The collagen-based double-layer membrane obtained is not transparent. It has an excellent mechanical stability, flexibility, good hemostatic properties, but only limited fluid absorption due to its very low porosity. The collagen layers are joined physically, the mechanical stability of the junction is very high. The material obtained can be used in both ex vivo and in vivo medical applications.

### Example 3

### Manufacture of a Multilayer Collagen-based Membrane-Like Material (Uncompressed Sponges+Compressed Sponges).

Two freeze-dried collagen sponges (Collatamp®, manufacturer: SYNTACOLL AG, Herisau, Switzerland) are conditioned at 21° C. in a moisture chamber to water content of 14%. After conditioning, the sponges are placed one above the other to form a double-layer and prepared for thermo-mechanical-pressing forming a double-layer construct as in Example 1.

On the top of this material, an additional freeze-dried collagen sponge (i.e. Collatamp®, manufacturer: SYNTACOLL AG, Herisau, Switzerland) is placed, forming a construct having two layers of previously pressed sponges, and a third layer of uncompressed sponge of defined porosity. This three-layer construct is then pressed to a membrane. Continuous heat of 100° C. and pressure of 25 kg/cm² is applied to the sponges for 10 seconds to form a new three-layer construct.

After finishing pressing, the pressure surfaces are opened without pre-cooling. The collagen-based double-layer membrane obtained is not transparent. It has an excellent mechanical stability, flexibility, and good hemostatic properties. The previously pre-pressed layer has a very limited fluid absorption, but the third (spongy) layer has an excellent fluid absorption and can absorb a fluid amount of up to 10 times its own weight. All collagen layers are joined physically, the mechanical stability of the junction is very high.

The material obtained can be used in both ex vivo and in vivo medical applications, especially as a wound covering material, and hemostatic material.

### Example 4

### Manufacture of a Multilayer Collagen-Based Membrane-Like Material (Uncompressed Sponges+Transparent Membrane)

A freeze-dried collagen sponge (Collatamp®, manufacturer: SYNTACOLL AG, Herisau, Switzerland) is conditioned at 21° C. in a moisture chamber to water content of 14%. On the top of this sponge, an air-dried transparent collagen membrane (Collatamp-Fascia®, manufacturer: SYNTACOLL AG, Herisau, Switzerland) is placed. The Fascia is conditioned to water content of 20%. This double-layer construct is then pressed to a membrane. Continuous heat of 100° C. and pressure of 25 kg/cm² is applied to the sponges for 10 seconds to form a new double-layer construct.

After finishing pressing, the surfaces or the press are opened without pre-cooling. The collagen-based double-membrane obtained is not transparent. It has an excellent mechanical stability, flexibility, and good hemostatic properties. The previously spongy layer has an excellent fluid absorption and can absorb a fluid amount of up to 10 times of its own weight. The previously fascia layer remains hemostatic, but absorbs fluids in only limited quantity. This layer serves both as mechanical and biological barrier of limited water and air permeability covering the surface of the product. Both collagen parts are joined physically, the mechanical stability of the junction is very high. The material obtained can be used in both ex vivo and in vivo medical applications, especially as a wound covering material, hemostatic material, and dressing for split- of full-skin donor sites.

### Example 5

### Manufacture of a Multilayer Collagen-Based Membrane-Like Material (Pre-Pressed Sponges Plus 4-Transparent Membranes)

Two freeze-dried collagen sponges (i.e. Collatamp®, manufacturer: SYNTACOLL AG, Herisau, Switzerland) are conditioned at 21° C. in a moisture chamber to water content of 14%. After conditioning, the sponges are placed one above the other to form a double-layer and prepared for thermo-mechanical pressing. This forms a double-layer construct as in Example 1.

On the top of this construct, an air-dried transparent collagen membrane (i.e. Collatamp-Fascia®, manufacturer: SYNTACOLL AG, Herisau, Switzerland) is placed. The Fascia is conditioned to water content of 20%. The two layers of this material consist of two previously pressed sponges, and the third one of a not pressed but flexible collagen membrane. This three-layer construct is then pressed to form a multilayer membrane. Continuous heat of 100° C. and pressure of 25 kg/cm² is applied to the construct for 10 seconds to form a tri-layer construct.

After finishing pressing, the press surfaces are opened without pre-cooling. The collagen-based triple-layer membrane obtained is not transparent. It has an excellent mechanical stability, flexibility, and good hemostatic properties. The previously spongy layer has lower fluid absorption than previously uncompressed, and can absorb a fluid amount of up to 10 times of its own weight. The previously fascia layer remains hemostatic, but absorbs fluids in only limited quantity. This layer serves both as mechanical and biological barrier of limited water and air permeability covering the surface of the product. Both collagen parts are joined physically, the mechanical stability of the junction is very high. The material obtained can be used in both ex vivo and in vivo medical applications, especially as a wound covering material, hemostatic material, and a dressing for split- of full-skin donor sites.

### Example 6

### Manufacture of a Multilayer Collagen-Based Material in the Form of Tubes of Channels With Open Lumen

Two freeze-dried collagen sponges (Collatamp®, manufacturer: SYNTACOLL AG, Herisau, Switzerland) are conditioned at 21° C. in a moisture chamber to water content of 14%. After conditioning, the sponges are placed one above the other to form a double-layer and prepared for thermo-mechanical pressing. This forms a double-layer construct as in Example 1.

This construct is then turned around a tube made from a non-adhesive, thermo-stable agent (i.e. medical grade paper) and thermally pressed to obtain a collagen-membrane tube. Continuous heat of 100° C. and pressure of 25 kg/cm² is applied to the sponges for 10 seconds. After finishing pressing, the press surfaces are opened without pre-cooling. The collagen-based tube obtained is not transparent. It has an excellent mechanical stability, flexibility, and good hemostatic properties. The central non-adhesive material can be easily removed directly after manufacturing or later, for example, directly before use. After wetting, the tubular construct has an excellent mechanical stability and flexibility. It can be used for guiding tissue reconstruction, which is for the reconstruction of tubular organs or nerves.

### Example 7

### Manufacture of a Multilayer Collagen-Based Material in the Form of Tubes or Channels in Which the Lumen is Filled by the Core made of a Collagen Material of Different Porosity

This exemplary product is made from freeze-dried and pre-pressed collagen membrane(s) and from a freeze-dried uncompressed collagen sponge of different porosity similar to that described in Example 6. The uncompressed sponge is covered on both sides by pre-pressed collagen membrane(s), wrapped about a tube of medical grade paper, and treated simultaneously with heat and pressure to form a tri-layer construct. Continuous heat of 100° C. and pressure of 25 kg/cm² is applied to the sponges for 10 seconds. After finishing pressing, the press surfaces are opened without pre-cooling. The collagen-based tube obtained is not transparent. It has an excellent mechanical stability, flexibility, and good hemostatic properties. The final material creates a construct with a core of different porosity. The core will absorb fluids up to 20× its own weight. A low-swelling collagen membrane protects the core. This construct can be used for reconstruction of missing tissue including bone and nerves.

### Example 8

### Manufacture of a Multilayer Collagen-Based Membrane-Like Material in Form of Tubes or Channels With Lumen Filled With a Core of a Collagen Material of Different Porosity, and With Longitudinally Oriented Channels

The final product is manufactured as described in Example 7, but additional longitudinally oriented channels are created in the core of the tube by incorporation of wire(s) of various diameters into the core material prior to manufacturing such tubes.

### Example 9

### Mechanical Strength Determination

This example measures the wet tensile strength, and suture retention limits, of multilayer collagen sheets of the present invention.

The multilayer collagen layers are prepared from reconstituted collagen sponges (Collatamp®, 10×10 cm) that have not been subjected to sterilization procedures, and that have been stored at room temperature and 13% relative humidity. These sponges have a spongy, porous surface and smooth skin surface. The prepress multilayer constructs are prepared by stacking one sponge on top of the other, with their skin surfaces abutting each other. Single layers and multilayer constructs are covered on their press plate sides with medical grade paper, placed in a hydrostatic press (Vogt GmbH, Berlin, Germany) pre-calibrated to 80 degrees C., and subjected to a uniform mechanical pressure of 5 kg/cm² (23 bar for 9.5×9.5) for 30 seconds.

Control materials tested are (1) uncompressed single layers of reconstituted collagen sponge (Collatamp®) and reconstituted collagen transparent membrane (Collatamp Fascie_), (2) compressed single layers of reconstituted collagen sponge (Collatamp®), and (3) the modified peritoneum-based collagen bilayer product, Bio-Gide®, manufactured and sold by Geistlich A G, and described in U.S. Pat. No. 5,837,278. The Bio-Gide sample characteristics are as follows: Batch: 010249, Expiration date: APR 2004, Dimensions: 22×28 mm, Packaging: Two nested molded trays with Tyvek peelable foil, Sterilization: Gamma Sterilization. The Bio-Gide product is a white membrane with one smooth and one rough side characterized by protruding fibers, is 0.42 mm thick, and weighs 120 mg (19.5 mg/cm2).

Test Methodology:

(1) Wet tensile strength: Test strips (8 mm×20 mm) of the samples are allowed to swell for at least 5 minutes in deionized water. A piece of cardboard is insert between the rubberized jaws of the universal testing machine and an initial pulling force of 0.2 newtons (N) applied to stretch the sample at a pulling speed of 60 mm/minute.

(2) Suture retention: Test strips (15 mm×30 mm) marked 5 mm from the edge of the small side and 7.5 mm from the edge of the long side. The test strips are allowed to swell for at least 5 minutes in deionized water. The unmarked small ends of the test strips are inserted (together with the layer of cardboard) into the jaws of the testing machine. A suture (Certilen EP 2, DS 18, braided thread, 0.2-0.25 mm, needle: 18 mm) is drawn through the mark, attached to the lower jaw, and the tear through force for the suture is determined. Each sample is analyzed five times. The initial pulling force is 0.1 newtons (N) and the sample is stretched at a speed of 60 mm/minute

The result of this testing is presented below in Table 1.

TABLE 1

| | Number of | | Wet tensile | | | |
|---|---|---|---|---|---|---|
| Sample | Layers | Thickness | strength (WTS) (N) | WTS/n | Suture retention | SR/n |
| No. | (n) | (mm) | (8 mm) | (N) | (SR) (max) (N) | (N) |
| 0* | 1 | 4.9 | 1.75 | 1.75 | 0.32 | 0.32 |
| 1 | 1 | 0.12 | 2.20 | 2.20 | 0.41 | 0.41 |
| 2 | 2 | 0.20 | 4.91 | 2.46 | 0.78 | 0.39 |
| 3 | 3 | 0.30 | 6.28 | 2.09 | 1.12 | 0.37 |
| 4 | 4 | 0.40 | 9.30 | 2.33 | 1.92 | 0.48 |
| 5 | 5 | 0.52 | 15.04 | 3.01 | 3.40 | 0.68 |
| Fascie | 1 | 0.03 | 2.87 | 2.87 | 0.22 | 0.22 |
| Bio-Gide | 1 | 0.42 | 7.63** | 7.63 | 5.28*** | 5.28 |

*Not compressed,
**single analysis,
***double analysis

The wet tensile strength of the compressed Collatamp sponge is higher than that of the uncompressed sponge, and is comparable to that of the Collatamp Fascie. A greater number of layers in the multilayer sheet material results in a higher WTS. A multilayer sheet having three layers of compressed sponge is comparable in WTS to the Bio-Gide product, while multilayer sheets with 4 and five layers exhibit a greater WTS that the Bio-Gide product.

The suture retention of the compressed sponge is improved relative to the uncompressed sponge and the Collatamp Fascie membrane. After puncturing by the needle, the Collatamp Fascie suffers from widespread and easy irregular tearing. The compressed sponge is much less sensitive in this regard and tears more uniformly. The multilayer sheets exhibit significantly better suture retention the greater the number of layers. The suture retention strength increases are greater than simply additive after the multilayer sheet exceeds three layers of compressed sponge.

### Example 10

### Interlayer Bonding Strength of Multilayer Sheets

This example determines the interlayer bonding strengths of multilayer sheets prepared at different temperatures and pressures under dry and wet conditions.

Conditioning of the Collatamp sponges: Collatamp® sponges (10×10 cm) that have, and have not, been sterilized by exposure to ethylene oxide (EO-sterilized), are removed from their packaging and conditioned for 1 hour at 25° C. and 50% r. h. in an environmental test chamber (Binder). Based on a dry weight determination, the moisture content of the samples is 14.4% for the non-sterile sponges and 15.14% for the ethylene-oxide sterilized sponges. Two sponges of the same type are combined into a bilayer construct with their skin surfaces abutting each other, placed in a medical grade paper bag protected by a plastic bag and the protected construct placed into an environmental chamber. A paper stripe 1 cm wide (0.04 mm thick) is placed at one edge between the double sponges to prevent the bonding of the collagen layers at the edge. This non-laminated edge served later as clamping point for measuring the delamination force. The time between removal from the environmental test chamber and thermal compression is kept as short as possible. Sample constructs are compressed in a hydrostatic press (Vogt GmbH, Berlin, Germany) pre-calibrated to different temperatures at two uniform mechanical pressure of 5 kg/cm² for 10 seconds.

Composite Bonding Evaluation: The bonding strength of the samples is evaluated with a tension-testing machine under both dry and wet conditions. Three different samples stripes 15 mm wide, including the one cm wide non-bonded area at one end, are cut out of each bilayer sheet. One sample strip from each bilayer sheet is examined under dry and wet conditions. Wet measurements are made after swelling the sample strip in de-ionized water at room temperature for more than 5 minutes. The two layers are clamped at their one cm detached edge, and force applied perpendicularly to the bilayer interface, at a speed of 3 mm/sec. The required force in newtons to separate the layers over the entire length (about 2×80 mm) is recorded.

TABLE 2A
Sheet Dry Bond Strength

| Compression | | | Ø pre- |
|---|---|---|---|
| temp (° C.) | Delamination behavior | Expenditure of force (N) | tear bonding (N) |
| Non-sterile | Sponge Layer Samples | | |
| 30 | Only destructive separation | Uneven | ˜0.05 |
| 40 | Only destructive separation | Uneven | ˜0.10 |
| 60 | Only destructive separation | Uneven | ˜0.15 |
| 80 | Only destructive separation | Uneven | ˜0.20 |
| 100 | Only destructive separation | Uneven | ˜0.90 |
| 120 | Only destructive separation | Uneven | ˜2.0 |
| 140 | Only destructive separation | Uneven | ˜1.0 |
| 160 | Only destructive separation | Uneven | ˜1.5 |
| EO-Sterilized | Sponge Layer Samples | | |
| 30 | Separation easy | 0.03-0.10, uniform | ˜0.05 |
| 40 | Separation easy | 0.04-0.14, uniform | ˜0.08 |
| 60 | Only destructive separation | 0.08->0.40, uneven | ˜0.20 |
| 80 | Only destructive separation | 0.08->0.40, uneven | ˜0.40 |
| 100 | Only destructive separation | 0.20-0.80, uneven | ˜0.50 |
| 120 | Only destructive separation | 0.60-1.20, uneven | ˜0.80 |
| 140 | Only destructive separation | 0.40-0.80, uneven | ˜0.60 |
| 160 | Only destructive separation | 0.40-1.20, uneven | ˜0.60 |
| 180 | Only destructive separation | 0.60-1.20, uneven | ˜1.0 |
| 200 | Only destructive separation | 0.60-1.40, uneven | ˜0.80 |

A significant increase of the bonding strength with increasing compression temperature is observed. The dry bond strength of the non-sterile bilayers prepared at 30° C. and above is such that only the initial de-lamination force could be measured, and the tear strength is measured after this initial determination. Bilayers prepared from sterile sponges at less than 60° C. are easily and uniformly separable. Bilayers prepared from sterile sponges at 60° C. and above tear. At 60° C. and above it is no longer possible to separate both layers non-destructively. During de-lamination one of the two layers is first subject to edge tearing and eventually tears off. The dry bonding strength of non-sterile bilayers prepared at 100° C. is uniformly very strong; both layers are inseparable. The bonding strength is higher than the tensile strength of the material. Non-sterile bilayers prepared at 120° C. and above are torn immediately and exhibit immediate edge tearing.

Table 2B below presents the results of the wet bond strength measurements.

TABLE 2B
Sheet Wet Bond Strength

| Compression | | | |
|---|---|---|---|
| temperature | | Expenditure of force | Ø Bonding |
| (° C.) | Delamination behavior | during delamination (N) | before tearing (N) |
| Non-sterile | Sponge Layer Samples | | |
| 30 | Uniform delamination | 0.05-0.10 | 0.07 |
| 40 | Uniform delamination | 0.05-0.08 | 0.07 |
| 60 | Uniform delamination | 0.03-0.07 | 0.05 |
| 80 | Uniform delamination | 0.02-0.04 | 0.03 |
| 100 | Only destructive separation | 0.08-0.20 | 0.12 |
| 120 | Uniform delamination | 0.12-0.24 | 0.16 |
| 140 | Only destructive separation | 0.12-0.18 | 0.16 |
| 160 | Only destructive separation | 0.12-0.22 | 0.16 |
| EQ-Sterilized | Sponge Layer Samples | | |
| 30 | Separation easy | 0.01-0.04, uniform | ˜0.03 |
| 40 | Separation easy | 0.01-0.06, uniform | ˜0.03 |
| 60 | Separation easy | 0.01-0.02, uniform | ˜0.02 |
| 80 | Separation easy | 0.01-0.03, uniform | ˜0.02 |
| 100 | Separation easy | 0.02-0.04, uniform | ˜0.03 |
| 120 | Separation easy, first | 0.04-0.16, uneven | ˜0.08 |
| | increase in bonding | | |
| 140 | Good bonding | 0.10-0.18, uniform | ˜0.12 |
| 160 | Only destructive separation | 0.14-0.18, uniform | ˜0.16 |
| 180 | Only destructive separation | 0.12-0.24, uniform | ˜0.18 |
| 200 | Only destructive separation | 0.12-0.22, uniform | ˜0.16 |

The wet bonding strength of bilayer sheets prepared from non-sterile sponges and EO-sterilized sponge layers increases rapidly starting at 100° C. and 120° C. respectively. Bilayers prepared from non-sterile sponge layers at 100° C. and above exhibit edge tearing, which prevents a complete de-lamination and causes an uneven expenditure of force. Nonetheless, the non-sterile bilayer prepared at 120° C. exhibits strong, uniform bond strength that permits uniform delamination.

Bilayers prepared from EO-sterilized sponge layers at 30 to 140° C. could be separated non-destructively. The bilayer bond strength of EO-sterilized sponges prepared at 140° C. is uniformly strong. Bilayers prepared at 160° C. and above are not able to be completely delaminated; resulting from edge tearing of one of the compressed sponge layers that eventually tears off.

For most of the samples, the expenditure of force increases towards the end of the measurement because the bonding is likely stronger at the outer rim than in the middle of the sponges.

### Example 11

### Multilayer Separation Susceptibility in Aqueous Medium

The bonding stability in neutral phosphate buffer of compressed tri-layer sheets prepared at differing compression temperatures from non-sterile Collatamp® sponges, are evaluated in comparison to the stability of the Bio-Gide PERIO (Geistlich) product described in Example 9 above.

Materials: 0.1 M phosphate buffer, pH 7.4, is prepared from 14.6 g Na₂HPO₄×H₂O and 2.48 g KH₂PO₄, diluted to 1000 ml with deionized and distilled water; three strips (10×30 mm) of each of eight tri-layer samples prepared in accordance with the process parameters described in Example 10 above; Bio-Gide PERIO Geistlich, batch: 010067, expiration date: January 2004, size: 16×22 mm, cut in half to 16×11 mm.

Procedure: Three samples each for a given compression temperature and the single sample of Bio-Gide are immersed at room temperature in separate 100 ml (id=40 mm) screw cap jars filled with 30 g of buffer (initial pH is 7.33). The jars are closed and allowed to stand at room temperature.

The jars are swirled to set the samples into a spinning motion. The samples are visually observed for any separation of the layers. After seven days, no sample is observed to exhibit spontaneous layer separation. After seven days, it is possible to disconnect single layers of the tri-layer sheets prepared at a temperature of up to 100° C. using mechanical force without destruction of particular layers. It is not possible to mechanically de-laminate the tri-layer sheets prepared at a temperature above 100° C. using mechanical force without layer destruction.

Multilayer materials of the present invention prepared at or below 100° C. may be used for implantation for tissue regeneration and/or implants that are sutured in place and not subjected to repetitive mechanical separation forces. In these applications, any separation tendency of these multilayer sheets is not pertinent to performance since in vivo mechanical forces are not applied to the product. In these cases, during the first week of implantation, sufficient tissue in-growth occurs to stabilize, and take advantage of the engineered multilayer structure of the present implant invention. Multilayer sheets prepared at about 120° C. or above are more appropriate for use in applications subject to repeated exposure to relatively high mechanical forces, for instance in tendon prostheses, abdominal wall patches, and the like. The observation record is presented in Table 3 below.

TABLE 3
Spontaneous de-lamination

| Sample | 30° C. | 40° C. | 60° C. | 80° C. | 100° C. | 120° C. | 40° C. | 160° C. | Bio-Gide |
|---|---|---|---|---|---|---|---|---|---|
| After 1 day | N | N | N | N | N | N | N | N | N |
| After 2 days | N | N | N | N | N | N | N | N | N |
| After 3 days | N | N | N | N | N | N | N | N | N |
| After 4 days | N | N | N | N | N | N | N | N | N |
| After 7 days | N | N | N | N | N | N | N | N | N |
| pH (day 7) | 7.28 | 7.27 | 7.28 | 7.30 | 7.29 | 7.32 | 7.30 | 7.32 | 7.28 |
| Mechanical | Y¹ | Y² | Y³ | Y⁴ | Y⁵ | Y⁶ | N⁷ | N⁸ | N⁹ |
| de- | | | | | | | | | |
| lamination*: | | | | | | | | | |
| Thickness | 1.0 | 0.9 | 0.7 | 0.6 | 0.6 | 0.6 | 0.5 | 0.6 | 0.8 |
| after 7 | | | | | | | | | |
| days in | | | | | | | | | |
| buffer (mm) | | | | | | | | | |

Legend
Y, N Y = layers separable; N = layers do not separate
*Using “hand force” - de-lamination by separation by fingers (no defined force)
¹ Very easy de-lamination, connection only by adhesion, no destruction of layers by de-lamination process
2-5 Easy de-lamination, connection only by adhesion; no destruction of layers by de-lamination process
⁶ Strong connection, de-lamination very difficult, separation test leads to destruction of material
^{7,8} Very strong connection, de-lamination not possible
⁹ Very strong adhesion, de-lamination not possible, leather-like material

### Example 11

### Physical and Fluid Absorption Measurements of Compressed Reconstituted Layers

Single layers of EO-sterilized and non-sterile Collatamp® sponges are compressed and their physical properties measured. Collatamp® sponges are prepared from an aqueous dispersion of 0.4% bovine collagen, include about 5.6 mg/cc collagen and have a dry thickness of about 5 mm.

Collatamp® sponges (10×10 cm) that have, and have not, been sterilized are removed from their packaging and conditioned for 1 hour at 25° C. and 50% r. h. in an environmental test chamber (Binder). Based on a dry weight determination, the moisture content of the samples is 17.4% for the non-sterile sponges and 14.4% for the ethylene-oxide sterilized sponges. Two sponges of the same type are placed in a medical grade paper bag protected by a plastic bag and the protected construct placed into an environmental chamber. The time between removal from the environmental test chamber and thermal compression is kept as short as possible. The single layer sponges are compressed in a hydrostatic press (Vogt GmbH, Berlin, Germany) pre-calibrated to different temperatures (30° C., 40° C., 60° C., 80° C., 100° C., 120° C., 140° C., 160° C.) at uniform mechanical pressures of 5 kg/cm² for 10 seconds, or 10 kg/cm2 for 10 seconds. The EO-sterilized compressed sponges are compressed only at 5 kg/cm2.

The compressed samples are weighed dry, then completely immersed in deionized water at room temperature and allowed to swell for a one-minute or one hour. The samples are retrieved vertically with two pairs of forceps and allowed to drain for 5 sec. The lower edge is stripped of water and weighed.

The dry and wet thickness and the amount of water absorbed by the compressed sponges are presented in Tables 4A and 4B below.

TABLE 4A
EO-Sterile Collatamp Sponge Layers (5 kg/cm2)

| | | | | | Water | | | |
|---|---|---|---|---|---|---|---|---|
| | | | | Wet Mass | uptake | Dry | Wet Mass 2 | Water |
| Compression | Dry | Wet | Dry | 1 (60 sec) | (mg/mg) | mass 2 | (60 min) | uptake |
| temp (° C.) | Thickness | Thickness | Mass 1 | (grams) | (60 sec) | (mg) | (Grams) | (mg/mg) (60 min) |
| 30 | 0.16 | 0.36 | 72 | 0.40 | 4.6 | 72 | 0.69 | 8.6 |
| 40 | 0.13 | 0.35 | 87 | 0.50 | 4.7 | 87 | 0.74 | 7.5 |
| 60 | 0.10 | 0.30 | 78 | 0.40 | 4.1 | 82 | 0.57 | 6.0 |
| 80 | 0.09 | 0.21 | 73 | 0.35 | 3.8 | 83 | 0.54 | 5.5 |
| 100 | 0.06 | 0.23 | 79 | 0.41 | 4.2 | 77 | 0.50 | 5.5 |
| 120 | 0.06 | 0.21 | 82 | 0.45 | 4.5 | 85 | 0.59 | 5.9 |
| 140 | 0.06 | 0.23 | 73 | 0.42 | 4.8 | 68 | 0.43 | 5.3 |
| 160 | 0.05 | 0.33 | 86 | 0.45 | 4.2 | 76 | 0.58 | 6.6 |
| 180 | 0.05 | 0.37 | 71 | 0.36 | 4.1 | 72 | 0.58 | 7.1 |
| 200 | 0.05 | 0.22 | 72 | 0.37 | 4.1 | 77 | 0.53 | 5.9 |

TABLE 4B
Non-Sterile Collatamp Sponge Layers (5 kg/cm² and 10 kg/cm²)

| | | | | | | | | |
|---|---|---|---|---|---|---|---|---|
| | | | | | Water | | | |
| | | | | Wet Mass − | uptake | Dry | Wet Mass − 2 | Water |
| Compression | Dry | Wet | Dry | 1 (60 sec) | (mg/mg) | mass − 2 | (60 min) | uptake |
| temp (° C.) | Thickness | Thickness | Mass − 1 | (grams) | (60 sec) | (mg) | (Grams) | (mg/mg) (60 min) |

Pressure 5 kg/cm²

| | | | | | | | | |
|---|---|---|---|---|---|---|---|---|
| 30 | 0.17 | 0.39 | 79 | 0.36 | 3.6 | 93 | 0.80 | 7.6 |
| 40 | 0.17 | 0.33 | 81 | 0.38 | 3.7 | 93 | 0.77 | 7.3 |
| 60 | 0.10 | 0.24 | 82 | 0.41 | 4.0 | 86 | 0.68 | 6.9 |
| 80 | 0.08 | 0.22 | 79 | 0.40 | 4.1 | 87 | 0.63 | 6.2 |
| 100 | 0.07 | 0.23 | 78 | 0.47 | 5.0 | 81 | 0.61 | 6.5 |
| 120 | 0.05 | 0.18 | 78 | 0.39 | 4.0 | 85 | 0.60 | 6.1 |
| 140 | 0.05 | 0.18 | 78 | 0.34 | 3.4 | 76 | 0.51 | 5.7 |
| 160 | 0.06 | 0.25 | 72 | 0.39 | 4.4 | 83 | 0.61 | 6.3 |

Pressure 10 kg/cm²

| | | | | | | | | |
|---|---|---|---|---|---|---|---|---|
| 30 | 0.12 | 0.29 | 80 | 0.33 | 3.1 | 89 | 0.62 | 6.0 |
| 40 | 0.11 | 0.22 | 80 | 0.40 | 4.0 | 92 | 0.63 | 5.8 |
| 60 | 0.08 | 0.21 | 85 | 0.39 | 3.6 | 87 | 0.53 | 5.1 |
| 80 | 0.06 | 0.21 | 79 | 0.34 | 3.3 | 85 | 0.55 | 5.5 |
| 100 | 0.06 | 0.22 | 85 | 0.40 | 3.7 | 82 | 0.48 | 4.9 |
| 120 | 0.07 | 0.22 | 77 | 0.39 | 4.1 | 80 | 0.53 | 5.6 |
| 140 | 0.06 | 0.21 | 76 | 0.44 | 4.8 | 80 | 0.54 | 5.8 |
| 160 | 0.05 | 0.20 | 70 | 0.38 | 4.4 | 87 | 0.51 | 4.9 |

The samples compressed with 5 kg/cm² remain opaque white up to a temperature of 160° C., while the samples compressed with 10 kg/cm² assume more of a parchment-like transparent appearance at temperatures of 100° C. and above. With increasing temperature, the compressed sponge samples thickness is increasingly reduced.

### Example 12

### Physical Measurements of Compressed Denser Collagen Sponges

(1) Single Sponge Measurement Processed Under Optimized Conditions

Collatamp® II sponges are prepared from a dispersion of 2.5% bovine collagen and have a density of about 30 mg/cc collagen. The Collatamp® II sponges used in this experiment include about 15% residual moisture (dry weight) and are not sterile. The Collatamp II sponges are preconditioned and compressed as described above for the Collatamp® sponges at a pressure of 10 kg/cm² at 80° C. for 10 seconds.

The compressed sponges are immersed in deionized water and the time for their complete swelling observed. The thermally compressed sponge was completely expanded in about 10 seconds, in comparison with an uncompressed Collatamp II sponge that took about two minutes to completely swell. Measurements of the water swelling behavior of the compressed and uncompressed Collatamp II sponges after ten and 90 seconds are presented in Table 5 below.

TABLE 5

| Test Sample: Collatamp II | Uncompressed | | Thermal compressed | |
|---|---|---|---|---|
| Swelling time (sec) | 10 | 90 | 10 | 90 |
| Sample weight (mg) | 335 | 355 | 343 | 326 |
| Water uptake (g) | 5.18 | 8.71 | 10.10 | 9.36 |
| Water uptake (mg/mg of sample) | 15.5 | 24.5 | 29.4 | 28.7 |
| Water uptake (% of max) | 53 | 84 | 100 | 100 |

(2) Properties of Compressed Dense Sponges Processed Under Varied Conditions

This example measures the physical properties of Collatamp® II sponges processed at various temperatures and pressures of 5 kg/cm² and 10 kg/cm².

Preparation of the samples: Collatamp® II sponges (10×10 cm) prepared from a dispersion of 2.5% equine collagen, having a density of about 30 mg/cc collagen and that have, and have not, been sterilized, are removed from their packaging and conditioned for 2 hours at 25° C. and 50% r. h. in an environmental test chamber (Binder). Based on a dry weight determination, the moisture content of the samples is between about 15.2 and 16.6%. Two sponges are placed in a medical grade paper bag protected by a plastic bag and the protected material placed into an environmental chamber. The time between removal from the environmental test chamber and thermal compression is kept as short as possible. The single layer sponges are compressed in a hydrostatic press (Vogt GmbH, Berlin, Germany) pre-calibrated to different temperatures (30° C., 40° C., 60° C., 80° C., 100° C., 120° C., 140° C., 160° C.) at uniform mechanical pressures of 5 kg/cm² for 10 seconds, or 10 kg/cm2 for 10 seconds. The compressed samples are weighed dry, then completely immersed in deionized water at room temperature and allowed to swell for a ten seconds or one hour before measurement. The samples are retrieved vertically with two pairs of forceps and allowed to drain for 5 sec. The lower edge is stripped of water and weighed.

The thickness of the compressed sponge, dry and wet, and the amount of water absorbed are presented in Table 6 below.

TABLE 6
Collatamp II Sponge Layers (5 kg/cm² and 10 kg/cm²)

| | | | | | Water | | | |
|---|---|---|---|---|---|---|---|---|
| | | | Dry | Wet Mass | uptake | Dry | Wet Mass 2 | Water |
| Compression | Dry | Wet | Mass 1 | 1 (10 sec) | (mg/mg) | mass 2 | (60 min) | uptake |
| temp (° C.) | Thickness | Thickness | (g) | (grams) | (10 sec) | (mg) | (Grams) | (mg/mg) (60 min) |
| Not | 5.0 | 4.5 | 0.40 | 8.97 | 21.4 | 0.38 | 12.22 | 31.2 |
| Compressed | | | | | | | | |

Pressure = 5 kg/cm²

| | | | | | | | | |
|---|---|---|---|---|---|---|---|---|
| 30 | 2.2 | 4.5 | 0.32 | 7.77 | 23.3 | 0.38 | 12.00 | 30.6 |
| 40 | 1.5 | 4.5 | 0.33 | 8.30 | 24.2 | 0.41 | 12.65 | 29.9 |
| 60 | 0.9 | 4.5 | 0.37 | 10.10 | 26.3 | 0.38 | 12.16 | 31.0 |
| 80 | 0.45 | 3.8 | 0.36 | 9.15 | 24.4 | 0.37 | 11.69 | 30.6 |
| 100 | 0.32 | 3.0 | 0.35 | 3.09 | 7.8 | 0.35 | 9.24 | 25.4 |
| 120 | 0.23 | 0.70 | 0.36 | 1.22 | 2.4 | 0.39 | 1.75 | 3.5 |
| 140 | 0.26 | 0.80 | 0.35 | 1.61 | 3.6 | 0.31 | 2.10 | 5.8 |
| 160 | 0.25 | 0.85 | 0.33 | 1.70 | 4.2 | 0.40 | 2.42 | 5.1 |
| 180 | 0.27 | 1.30 | 0.38 | 2.29 | 5.0 | 0.40 | 3.05 | 6.6 |
| 200 | 0.24 | 0.80 | 0.30 | 1.45 | 3.8 | 0.38 | 1.94 | 4.1 |

Pressure = 10 kg/cm²

| | | | | | | | | |
|---|---|---|---|---|---|---|---|---|
| 30 | 1.80 | 4.5 | 0.35 | 10.35 | 28.6 | 0.38 | 12.57 | 32.1 |
| 40 | 1.10 | 4.5 | 0.33 | 9.00 | 26.3 | 0.36 | 11.17 | 30.0 |
| 60 | 0.60 | 4.5 | 0.33 | 7.68 | 22.3 | 0.32 | 10.09 | 30.5 |
| 80 | 0.30 | 3.5 | 0.36 | 7.41 | 19.6 | 0.33 | 7.98 | 23.2 |
| 100 | 0.28 | 1.0 | 0.33 | 1.29 | 2.9 | 0.35 | 2.27 | 5.5 |
| 120 | 0.26 | 0.65 | 0.33 | 1.09 | 2.3 | 0.40 | 1.91 | 3.8 |
| 140 | 0.20 | 0.55 | 0.35 | 0.74 | 1.1 | 0.35 | 1.45 | 3.1 |
| 160 | 0.18 | 0.50 | 0.32 | 0.95 | 2.0 | 0.30 | 1.34 | 3.5 |
| 180 | 0.17 | 0.50 | 0.30 | 0.72 | 1.4 | 0.33 | 1.28 | 2.9 |
| 200 | 0.17 | 0.55 | 0.34 | 0.71 | 1.1 | 0.37 | 1.41 | 2.8 |

Sponges that are compressed with 5 kg/cm² at a temperature of 140° C. and above begin to assume a parchment-like condensed appearance with vitreous spots. Sponges that are compressed at 10 kg/cm² a temperature of 120° C. and above assume a similar appearance. Beginning with 140/160° C. the swollen samples have a leathery consistency and the sponges' color changes from white to yellow.

Water swelling capacity at 10 seconds decreases rapidly for samples prepared at 10-kg/cm2 beginning with a temperature of 100° C. The one-hour water absorption capacity of samples prepared at 100° C. (5kg/cm2) reflect slower water absorption, although the samples absorb at least about 75% of the water absorbed by the uncompressed sponge in one hour. Above 100° C., the one-hour capacities correlate to the 10-second capacity. At and below these temperature and pressure parameters, the compressed sponges are capable of recovering at least two thirds of their original thickness.

### Example 13

### Biological Properties

This example measures the biological properties, and specifically the hemostatic relevant properties, of Collatamp® II sponges processed at various temperatures and a pressure of 10 kg/cm². The aggregation of platelets is measured to determine the hemostatic properties and extent, if any, of collagen denaturation present in the processed sponges. A delay in platelet aggregation is believed to correspond to the presence of amounts of denatured collagen.

Preparation of the samples: Collatamp® II sponges (10×10 cm) that have not, been sterilized, are removed from their packaging and conditioned for 2 hours at 25° C. and 50% r. h. in an environmental test chamber (Binder). Based on a dry weight determination, the moisture content of the samples is 15.2%. Two sponges are placed in a medical grade paper bag protected by a plastic bag and the protected material placed into an environmental chamber. The time between removal from the environmental test chamber and thermal compression is kept as short as possible. The single layer sponges are compressed in a hydrostatic press (Vogt GmbH, Berlin, Germany) pre-calibrated to different temperatures (30° C., 40° C., 60° C., 80° C., 100° C., 120° C., 140° C., 160° C.) at uniform mechanical pressures of 10 kg/cm2 for 10 seconds.

Measurement of Platelet Aggregation

A sample of the compressed sponge is shredded and homogenized. A measured portion (about 29.4 mg) is dispersed in sterile aqueous buffer and an amount of dispersion providing 10 μg (1 ml) introduced into the Aggregometer (APACT). Samples that exhibit reduced platelet aggregation are retested using a 50-μg (1 ml) sample. The standard solubilized collagen reference is a 1-μg sample results in 8⁹% aggregation in 46 seconds. The results of the testing are presented in Table 7 below.

TABLE 7
Platelet Aggregation Thermally-Compressed Collatamp II

| | Homogenized | Aggregation (10 | | Aggregation (50 | |
|---|---|---|---|---|---|
| Compression | Sample | μg) | | μg) | |
| tempera- | Weight | % of | T | % of | T |
| ture (° C.) | (mg) | Max | (sec) | Max | (sec) |
| Uncompressed | 29.4 | 87 | 46 | — | — |
| 30 | 29.6 | 86 | 82 | — | — |
| 40 | 29.3 | 86 | 61 | — | — |
| 60 | 29.8 | 88 | 68 | — | — |
| 80 | 29.0 | 89 | 67 | — | — |
| 100 | 29.6 | 86 | 64 | 89 | 43 |
| 120 | 29.1 | 86 | 96 | 91 | 55 |
| 140 | 29.6 | n. a. | — | 88 | 91 |

Samples processed at temperatures at and below 120° C. are finely homogenized. Beginning with the sample prepared at 140° C., a fine homogenization and thus even distribution in the liquid is not possible; these samples are increasingly dispersed in the form of large particles.

Up to a compression temperature of 100° C., no negative effect on platelet aggregation is observed. The samples processed at 120° C. exhibit the first signs of a visible delay in the aggregation, while aggregation is still detectable with 50 μg samples prepared at 140° C. Consequently, samples prepared at 140° C. still retain collagen in substantially native form. The altered homogenization properties of these higher temperature-processed samples, which altered properties are observed during sample preparation, correlate to the observations of delayed platelet aggregation. Therefore, the test procedure, which is designed for testing solubilized collagen, reflects delays in aggregation based on particle size differences among samples.

### Example 14

### Tortellini Constructions and Release Profiles

Tortellini constructs according to the present invention are prepared employing a multilayer sponge core containing gentamycin sulfate. The following core constructs are prepared:

TC (a)—A single 5×5 cm sponge containing 200 mg of gentamycin.

TC (b)—Two compressed 5×5 cm sponges. each layer containing 100 mg of gentamycin.

TC (c)—Four compressed 5×5 cm sponges. each layer containing 50 mg of gentamycin.

Constructs TC (a), TC (b) and TC (c) are thermally pressed together with a pressure of 10 kg/cm2 at 80° C. for 10 seconds and then placed between two 10×10 cm Collatamp-I sponges and compressed together to made a sandwich with the core. The sandwich compression step is performed using a pressure of 45 kg/cm2 at room temperature for 10 seconds. A second compression step is performed where the edges or borders (ca. 2.0-2.5 cm, without touching the core) of the multilayer construct are thermally compressed have been thermally compressed at a pressure of 10 kg/cm2, 80° C. for 10 seconds. The TC samples are immersed in aqueous buffer solution and the amount of gentamycin released measured over time. The results are present in Table 8 below.

TABLE 8
Gentamycin sulfate released from each type of tortellini

| | | |
|---|---|---|
| TC (a) | TC (b) | TC(c) |

| Time (h) | (mg) | (%) | (mg) | (%) | (mg) | (%) |
|---|---|---|---|---|---|---|
| 0 | 0 | 0 | 0 | 0 | 0 | 0 |
| 0.5 | 27.4 | 13.7 | 11.8 | 5.9 | 18.4 | 9.2 |
| 1 | 65.8 | 32.9 | 37.6 | 18.8 | 38.8 | 19.4 |
| 2 | 147.6 | 73.8 | 109.8 | 54.9 | 74.6 | 38.3 |

Variations of this tortellini construct are described in the following Table 9.

TABLE 9
Tortellini Constructions

| | | Thickness of the | Thickness of core gentamycin- |
|---|---|---|---|
| | Outer Collagen | border laminate | containing area (outer layers + 4 |
| (TC) Sample ID | Sponges | (mm) | Collatamp-G layers) (mm) |
| TC-I | 2 × 1 | 0.17 | 1.1 |
| (one outer layer) | | | |
| TC-II | 2 × 2 | 0.30 | 1.8 |
| (two outer layers) | | | |
| TC-III (three outer layers) | 2 × 3 | 0.40 | 2.5 |

The tortellini constructs described in Table 9 consist of an outer skin made up of from one to three layers (upper and lower covers) of collagen sponges that do not contain gentamycin (Collatamp I-10×10cm). The core consists of four layers of Collatamp G (5×5 cm), each layer containing 50 mg of gentamycin sulfate. Each TC construct contains a minimum of six layers of collagen sponge.

The TC samples are immersed in aqueous buffer solution and the amount of gentamycin released measured over time. The results are present in Table 10 below.

TABLE 10
Gentamycin Release from Tortellini

| | | Gentamycin | Gentamycin | Control - release data for single |
|---|---|---|---|---|
| | | released | released | layer of uncompressed |
| TC Sample | Time (min) | (mg) | (%) | Collatamp-G (%) |
| I | 5 | 8.8 | 4 | 48 |
| I | 10 | 9.8 | 5 | 57 |
| I | 30 | 7 | 3 | 99 |
| I | 60 | 25 | 12 | 102 |
| I | 120 | 84.8 | 41 | — |
| II | 60 | 0.8 | 0 | 102 |
| III | 60 | 2.6 | 1 | 102 |

Sample TC II and III do not begin to release measurable amounts of gentamycin until about 60 minutes.

We claim:

1. A multilayer biocompatible sheet material comprising a first and second layer comprising reconstituted matrices of biocompatible collagen, which layers are physically adhered along at least a portion of a surface of each of said layers, wherein said material has sufficient flexibility to form tubes useful for tissue and organ reconstruction, and wherein at least one said layer is capable of absorbing sufficient fluids to form an expanded matrix capable of promoting cell growth.

2. A multilayer biocompatible sheet material according to claim 1 wherein said matrix is porous and capable of promoting formation of granulation tissue, angiogenesis and vascularization.

3. A sheet material according to claim 1 wherein said layers remain adhered to each other in contact with blood and tissue fluids under physiological conditions.

4. A sheet material according to claim 3 wherein said collagen of each layer exhibits the hemostatic and non-antigenic properties of native collagen.

5. A sheet material according to claim 4 having a wet tensile strength useful in surgical suturing.

6. A sheet material according to claim 5 wherein said wet tensile strength is measured to be about 5 N to about 25 N.

7. A sheet material according to claim 6 wherein said sheet material further comprises from one to four additional layers each comprising a reconstituted matrix of biocompatible collagen.

8. A sheet material according to claim 5 wherein said sheet material further comprises a third layer comprising a biocompatible synthetic polymer.

9. A sheet material according to claim 5 wherein said first layer expands about 3 to about 10 times in volume on contact with fluids.

10. A sheet material according to claim 9 wherein said second layer expands less than about 3 times in volume on contact with fluids.

11. A sheet material according to claim 9 wherein said first layer has a thickness of less than about 1 mm, and a density of about 250 mg/cc to about 500 mg/cc.

12. A sheet material according to claim 9 wherein said first and second layers have substantially the same dry thickness.

13. A sheet material according to claim 9 wherein said first and second layers have substantially different dry thickness and densities.

14. A tubular article having an inner and outer surface, comprising a sheet material according to claim 9 wherein said first layer comprises said inner surface.

15. A tubular article according to claim 14 wherein said outer surface comprises said second layer that expands less than about 2 times in volume on contact with fluids.

16. A tubular article according to claim 14 wherein said outer surface is water permeable and substantially non-porous.

17. A sheet material according to claim 6 wherein the adhesive strength of between said layers is about equal to or greater than the wet tensile strength of each of said layers.

18. A sheet material according to claim 17, wherein each layer comprises a compressed sponge or transparent membrane of reconstituted collagen.

19. A process for the preparation of a multilayer biocompatible sheet material having sufficient flexibility to form tubes useful for tissue and organ reconstruction, comprising
(a) aligning the surface of at first sheet of reconstituted matrix of biocompatible collagen with the surface of a second sheet of reconstituted matrix of biocompatible collagen to form a non-adherent bilayer construct, and
(b) applying mechanical pressure and elevated temperature simultaneously and uniformly along the entirety of said contacting surfaces of said construct for a time sufficient to adhere said layers but insufficient to denature said collagen.

20. A process according to claim 19, wherein said pressure is about 5 to about 10 kg/cm2, said elevated temperature is about 50 to about 120 degrees C., and said time is about 0.1 to about 60 seconds.

21. A process according to claim 20, wherein said first sheet comprises a biocompatible collagen sponge having a thickness of about 1 to about 10 mm, and a density of about 2 to about 60 mg/cc.

22. A process according to claim 21 wherein said second layer comprises a biocompatible collagen sponge having a thickness of about 1 to about 10 mm, and a density of about 2 to about 60 mg/cc.

23. A process according to claim 22 wherein said second layer comprises a biocompatible transparent collagen membrane having a thickness of about 0.01 to about 0.1 mm.

24. A process according to claim 20 wherein one or more additional layers of biocompatible reconstituted collagen matrix are aligned with said first and second layers to form a non-adherent construct.

25. A process according to claim 24 wherein said one or more additional layers are aligned subsequent to the application of heat and pressure to said first and second layers.

26. A multilayer biocompatible sheet material having sufficient flexibility to form tubes useful for tissue and organ reconstruction prepared according to the process of claim 19.

27. A multilayer sheet material according to claim 26 wherein the thickness of said first layer is reduced to about 1 to about 30 percent of its original thickness.

28. A multilayer sheet material according to claim 27 wherein the thickness of said first layer is reduced to about 1 to about 3 percent of its original thickness, and is capable of absorbing about 3 to about 7 times its weight in fluids in about sixty seconds.

29. A multilayer sheet material according to claim 28 wherein the thickness of said first layer is reduced to about 5 to about 30 percent of its original thickness, and is capable of absorbing about 4 to about 30 times its weight in fluids in about sixty seconds.

30. A multilayer sheet material according to claim 29 wherein the thickness of said first layer is reduced to about 6 to about 25 percent of its original thickness, and is capable of absorbing about 25 to about 30 times its weight in fluids in about sixty seconds.

31. A multilayer material according to claim 1, wherein one layer used for manufacturing is a sponge or a membrane containing 0.5-5 weight/volume % of collagen.

32. A multilayer material according to claim 31, wherein collagen layers have been previously simultaneously treated with heat and pressure.

33. A multilayer material according to 1 further comprising biologically active substances, selected from the group consisting of hemostatic agents, growth factors, cytokines, hormones, antibiotics, anti-inflammatory agents.
